(19) United States
(12) Patent Application Publication
(10) Pub. No.: US 2005/0159442 A1
(43) Pub. Date: Jul. 21, 2005

(54) Protein tyrosine phosphatase 1B (PTP-1B) inhibitors and methods of using the same

(76) Inventors: Danlin Xu, Piscataway, NJ (US); Francis Farrell, Buckingham, PA (US); Eugene B. Grant, Flemington, NJ (US); Frank K. Brown, Whitehouse Station, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/975,813

(22) Filed: Oct. 28, 2004

Related U.S. Application Data
(60) Provisional application No. 60/515,149, filed on Oct. 28, 2003.

(51) Int. Cl. A61K 31/4745

(52) U.S. Cl. 514/300

(57) ABSTRACT

A novel class of compounds has been identified that inhibit PTP-1B through a newly discovered binding interaction. Methods of treating, preventing or delaying the onset of, conditions mediated by PTP-1B are also provided. The compounds are capable of interacting with the primary binding pocket of PTP-1B via a hydrogen bond acceptor and have an aqueous phase free energy more positive than about −1200 kJ/mol. Representative compounds in the class are those of Formula I: 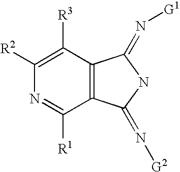
or an optical isomer, enantiomer, diastereomer, racemate or racemic mixture thereof, ester, prodrug or metabolite form, or a pharmaceutically acceptable salt thereof, wherein R¹, R² and/or R³ are independently hydrogen atoms or alkyl groups and G¹ and G² are aryl, heteroaryl, alkyl, arylalkanyl, arylalkenyl, arylalkynyl, heteroarylalkanyl, heteroarylalkenyl, or heteroarylalkynyl moieties.

The present invention claims priority from U.S. Provisional Patent Application Ser. No. 60/515,149 entitled “Protein Tyrosine phosphatase 1B (PTP-1B) inhibitors and methods using the same” filed Oct. 28, 2003, the contents of which are hereby incorporated by reference in their entirety. The present invention relates to pharmaceutical compositions comprising a novel class of PTP-1B inhibitors, as well as the use of these compositions to inhibit the activity of PTP-1B, as may be useful in the treatment or prevention of PTP-1 B mediated conditions.

### FIELD OF THE INVENTION

### BACKGROUND OF THE INVENTION

Protein-tyrosine phosphatases (PTPs) are a large family of transmembrane enzymes that dephosphorylate substrates involved in a variety of regulatory processes. (Fischer et al., Science, 1991, 253: 401-406). Protein tyrosine phosphatase-1B (PTP-1B) is the prototypic member of the PTP family. It was first purified from human placenta as a catalytic domain of 37 kDa (Tonks, et al., J. Biol. Chem., 1988, 263: 6722-6730) and was subsequently shown to occur in vivo as a full-length protein of 50 kDa (see e.g., Guan, et al., Proc. Natl. Acad. Sci. U.S.A., 1990, 87: 1501-1505). PTP-1B is composed of an N-terminal catalytic domain fused to a non-catalytic, C-terminal segment that serves a regulatory function, targeting the protein to the cytoplasmic face of membranes of the endoplasmic reticulum. PTP-1B is present in abundant amounts in various human tissues (Charbonneau et al., Proc. Natl. Acad. Sci. USA, 1989, 86: 5252-5256).

Recently, a number of observations have been made regarding PTP-1B levels in living. PTP-1B has been reported to act as a negative regulator of signaling events initiated by several growth factor/hormone receptor protein tyrosine kinases (PTKs), including the epidermal growth factor, as well as signaling events induced by cytokines. It has also been reported that PTP-1B may have regulatory roles in cancer development. PTP-1B has been shown to dephosphorylate p130^{Cas}, suggesting that it may have a regulatory role in mitogen-mediated signal transduction pathways (Liu et al., J. Biol. Chem., 1996, 271: 31290-31295). Evidence has also been provided that shows that PTP-1B may function as a specific, negative regulator of the p210 Bcr-Abl oncoprotein, a PTK that is directly responsible for the initial manifestations of chronic myelogenous leukemia (CML) (Kenneth et al., Mol Cell Biol, 1998, 18: 2965-2975). Overexpression of PTP-1B has further been reported as being potentially involved in p-185^{c-erb B2}-associated breast and ovarian cancers (Weiner et al., J. Natl. Cancer Inst., 1996, 86: 372-378). Of particular interest has been the compelling linkage between PTP-1B and insulin signaling defects associated with diabetes and other glucose-related, blood disorders. It has now been demonstrated that PTP-1B negatively regulates the insulin signaling pathway (Katsuya, et al., J. Biol. Chem., 2001, Vol. 276: 10207-10211) and suggested that inhibitors of PTP-1B may be useful in the treatment of Type 2 diabetes (Kennedy et al., Science, 1999, 283: 1544-1548).

Due at least in part to the voluminous literature linking PTP-1B levels to such a wide variety of health conditions, considerable interest has been focused on developing mediators of PTP-1B. Early design efforts were directed to peptidyl structures in which the labile phosphotyrosine (pTyr) residue is replaced with a nonhydrolyzable pTyr mimetic (Burke, et al., Biopolymers, 1998, 47: 225-241). One representative example of these mimetics was the difluoromethylphosphonic acid (DFMP) group. (Burke et al., Biochem. Biophys. Res. Commun., 1994, 204: 129-134, and Chen et al., Biochem. Biophys. Res. Commun., 1995, 216: 976-984). Certain peptides bearing difluoromethylphosphono-phenylalanine (F₂Pmp), and tripeptides bearing a cinnamic acid moiety were identified thereafter as PTP-1B inhibitors. Peptides bearing sulfotyrosine, O-malonyltyrosine, fluoro-O-malonyltyrosine, and other pTyr mimetics were also identified as inhibitors of PTP-1B (Jia et al., J. Med. Chem. 2001, 44: 4584-4594).

Because of the problems associated with the development of peptide-based therapeutics, more recent efforts have been focused on developing small-molecule PTP-1B inhibitors. As a result of these efforts, some small molecule PTP-1B inhibitors have been identified. Based upon three-dimensional modeling of the interaction of the identified small molecule inhibitors and PTP-1B, a phosphate group and at least one aryl group were identified as relevant to the interaction. (Burke, Bioorg. Med. Chem. 1998, 6: 1799-1810; Jia et al., J. Med. Chem. 2001, 44: 4584-4594). In particular, the phosphate and aryl groups were found to bind to the primary binding pocket of PTP-1B with high affinity and to a second noncatalytic aryl phosphate binding site with lower binding affinity.

Although many inhibitors of PTP-1B have been proposed, due to the prevalence of PTP-1B mediated conditions and diseases, additional PTP-1B inhibitors would yet desirably be available for use in the treatment of PTP-1B mediated conditions.

### SUMMARY OF THE INVENTION

The present invention provides a novel class of PTP-1B inhibitors that are believed to interact with PTP-1B via a newly discovered mechanism. More particularly, it has now been discovered that small molecules comprising a hydrogen bond acceptor group and having a more positive aqueous phase free energy are capable of inhibiting PTP-1B as is believed to occur via binding with high affinity to the primary binding pocket of PTP-1B. Compounds according to Formula I are representatives: 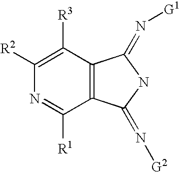
or an optical isomer, enantiomer, diastereomer, racemate or racemic mixture thereof, ester, prodrug or metabolite form, or a pharmaceutically acceptable salt thereof, as a PTP1B inhibitor, wherein R¹ and R², and/or R³ may be any monovalent substituent. Preferably, R¹ and R², and R³ will be selected so as to not unduly cause more negative aqueous phase free energy of the inventive compounds. More preferably, R¹, R², and/or R³ may independently be hydrogens or alkyl groups, whether straight, branched or cyclic such as but not limited to methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl, cyclohexyl and 2-methylpentyl. G1 and G2 may be any alkyl or aryl including moiety, wherein suitable alkyl moieties include, but are not limited to methyl, ethyl, n-propyl, n-butyl, isobutyl, sec-butyl, t-butyl, n-pentyl, 3-(2-methyl)butyl, 2-pentyl, 2-methylbutyl, neopentyl, n-hexyl, 2-hexyl, cyclohexyl and 2-methylpentyl, and suitable aryl moieties include, but are not limited to, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl, pyridinyl, pyrazinyl, pyrimidinyl, pyridazinyl, pyrrolyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, N-oxo-pyridyl, 1,1-dioxothienyl, benzothiazolyl, benzoxazolyl, benzothienyl, quinolinyl-N-oxide, benzimidazolyl, benzopyranyl, benzisothiazolyl, benzisoxazolyl, benzodiazinyl, benzofurazanyl, indazolyl, indolizinyl, benzofuryl, cinnolinyl, quinoxalinyl, pyrrolopyridinyl, furopyridinyl (such as furo[2,3-c]pyridinyl, furo[3,2-b]pyridinyl, or furo[2,3-b]pyridinyl), imidazopyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, thienothienyl and the like. Preferably G¹ and G² may be independently selected from (4-arylC₁₋₈alkyl)C₁₋₈alkyl. Particularly preferred compounds may thus be represented by Formula II:

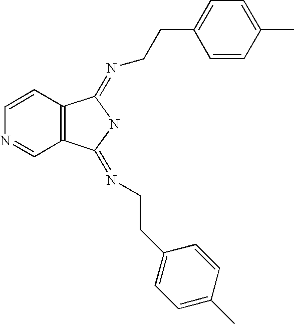

The present inventive compounds are readily and easily synthesized by those of ordinary skill in the art by application of known methodologies, and exhibit efficacy in inhibiting PTP-1B in vitro. As such, and in a first aspect, the present invention provides a pharmaceutical composition comprising an effective amount of one or more of the compounds of Formula I in combination with at least one pharmaceutically acceptable carrier. The pharmaceutical composition may either be adapted for administration for prophylactic purposes, i.e., to prevent or delay the onset of a PTP-1B mediated condition or disease, in which case a prophylactically effective amount of the inventive compound may be provided, or, may be adapted for administration for therapeutic purposes, i.e., to treat an existing PTP-1B mediated condition, in which case a therapeutically effective amount of the inventive compound may be provided. As used herein, the phrase “prophylactically effective amount” refers to an amount of the inventive compound that prevents or delays in a subject the onset of a disorder mediated by PTP-1B. The phrase “therapeutically effective amount” refers to an amount of the inventive compound that elicits the biological or medicinal response in a tissue system, animal or human that is being sought by a researcher, veterinarian, medical doctor or other clinician, which includes control or alleviation of the symptoms of a PTP-1B mediated disease or condition being treated. Advantageously, the pharmaceutical composition may be adapted for combination therapy, either by including an additional therapeutic agent, or by including directions for a dosing regimen including administration of a further pharmaceutical composition comprising an additional therapeutic agent.

Due at least in part to the high binding affinity with which the compounds of the present invention bind to PTP-1B, thereby inhibiting the activity of the same, the present invention also provides methods of inhibiting PTP-1B. Specifically, the method comprises causing an inhibitory amount of one or more compounds of the present invention, comprising a hydrogen bond acceptor and having a more positive aqueous free energy, to contact a medium comprising PTP-1B. In certain embodiments, the medium can be a cell.

The ability to inhibit PTP-1B is expected to be useful in delaying treating or preventing diseases or conditions mediated by PTP-1B, and as such the present invention further provides such methods. More particularly, the present invention provides a method of treating a subject suffering from, or delaying the onset of, a condition mediated by PTP-1B by administering a therapeutically or prophylactically, respectively, effective amount of a compound according to the present invention. Many such conditions have been identified, and all of these, as well as any that may be identified in the future, are considered to be within the scope of the invention.

Currently identified conditions known or believed to be mediated by PTP-1B may include, but are not limited to disorders in glucose and lipid metabolism, such as type I diabetes, type II diabetes, impaired glucose tolerance, insulin resistance, obesity, hyperlipidemia, hypertriglyceridemia, hypercholesterolemia, dyslipidemia or low HDL levels; immune dysfunctions including inflammatory bowel disease and autoimmunity diseases; dysfunctions of the coagulation system; allergic diseases including asthma; osteoporosis, proliferative disorders including cancers such as prostate cancer, ovarian cancer, leukemias, adipose cell tumors, adipose cell carcinoma, liposarcoma, and breast cancer, diseases with increased or decreased syntheses of effects of growth factors, diseases with decreased or increased synthesis of growth factors or cytokines that regulate the release of/or response to growth factors, disease of the brain including Alzheimer's disease and schizophrenia; infectious diseases; nephropathy; neuropathy; retinopathy; artherosclerosis; vascular restenosis; pancreatitis; polycystic ovary syndrome, ischemia, hypertension, stroke, and heart disease. Due to the well documented linkage between PTP-1B and insulin signaling defects, the methods of the present invention are particularly advantageously applied for the treatment of diabetes and other conditions commonly associated therewith, e.g., obesity, artherosclerosis, and the like.

### DETAILED DESCRIPTION OF THE INVENTION

The embodiments of the present invention described below are not intended to be exhaustive or to limit the invention to the particular embodiments disclosed in the following detailed description. Rather, the embodiments are described so that others skilled in the art can understand the principles and practices of the present invention.

The present invention relates to the discovery of a new class of small molecule inhibitors for protein tyrosine phosphatase 1B (PTP-1B), and uses of these inhibitor(s) in methods of inhibiting PTP-1B and/or for therapeutic or prophylactic treatment of conditions mediated by PTP-1B. Surprisingly, the newly discovered compounds do not necessarily contain a phosphate group or a DFMP group, previously identified as critical to binding with the primary binding pocket of PTP-1B and thereby inhibiting PTP-1B activity. Rather, the inventive compounds have a more positive aqueous phase free energy and comprise a hydrogen bond acceptor that strongly interacts with the primary binding pocket of PTP-1B. Although the phosphate groups in previously identified small-molecule PTP-1B inhibitors can form from 6 to 8 hydrogen bonds in the binding pocket, these inhibitors exhibit a more negative aqueous free energy, so that the overall binding affinity of these inhibitors with the binding pocket of PTP-1B is only moderate. To the contrary, even though the hydrogen bond accepting nitrogen in the present compounds may form only 2 hydrogen bonds with the PTP-1B binding pocket, the compounds have a much more positive aqueous free energy, so that the overall binding affinity of the inventive compounds with PTP-1B can be equivalent to, or even stronger than, that seen with previously identified small molecule inhibitors comprising a phosphate group.

Having discovered this new binding interaction, structurally distinct from that previously used in the design of small molecule inhibitors of PTP-1B, any compound(s) capable of interacting with PTP-1B in this fashion are considered to be within the scope of the present invention. More specifically, any compound having a more positive aqueous phase free energy and further comprising a hydrogen bond acceptor, is considered to be within the scope of the present invention.

As used herein “more positive aqueous phase free energy” is meant to indicate an aqueous phase free energy more positive than −1200 kJ/mol, preferably more positive than −460 kJ/mol, more preferably more positive than −60 kJ/mol, as may be calculated using 6-31G ESP charges, OPLS AA VDW and GBS implicit water model. Such calculation methods are known to those of ordinary skill in the art. Further, as used herein, a “hydrogen bond acceptor” can be any compound, or moiety thereof, capable of interacting with a positively charged compound or moiety via hydrogen bonding. Typical hydrogen bond acceptors may be strongly electronegative in nature and exemplary electronegative elements capable of this type of interaction include, but are not limited to, nitrogen, oxygen or fluorine, while exemplary moieties include nitrous groups, etc. Although phosphate groups are hydrogen bond acceptors, the inventive compounds also desirably exhibit a more positive aqueous phase free energy than can be exhibited by a compound comprising such moieties. Because the phosphate groups can cause the aqueous phase free energy of the compound to be, e.g. more negative than −1200 kJ/mol, resulting in weaker interaction between the compound and the PTP-1B, the inclusion of phosphate groups in the inventive compounds may be disfavored.

Generally speaking, the present inventive compounds can include those that are represented by Formula I: 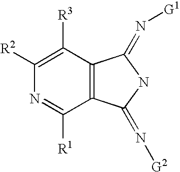
or an optical isomer, enantiomer, diastereomer, racemate or racemic mixture thereof, ester, prodrug or metabolite form, or a pharmaceutically acceptable salt thereof, as a PTP1B inhibitor, wherein R¹ and R², and/or R³ may be any monovalent substituent. Preferably, R¹ and R², and R³ will be selected so as to not unduly result in a more negative aqueous phase free energy in the inventive compounds. More preferably, R¹, R², and/or R³ can independently be hydrogens or alkyl groups, whether straight, branched or cyclic, including, but not limited to, methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl, cyclohexyl and 2-methylpentyl. G1 and G2 can be any alkyl or aryl moiety, wherein suitable alkyl moieties include, but are not limited to, methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl, cyclohexyl and 2-methylpentyl, and, suitable aryl moieties include, but are not limited to, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl, pyridinyl, pyrazinyl, pyrimidinyl, pyridazinyl, pyrrolyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, N-oxo-pyridyl, 1,1-dioxothienyl, benzothiazolyl, benzoxazolyl, benzothienyl, quinolinyl-N-oxide, benzimidazolyl, benzopyranyl, benzisothiazolyl, benzisoxazolyl, benzodiazinyl, benzofurazanyl, indazolyl, indolizinyl, benzofuryl, cinnolinyl, quinoxalinyl, pyrrolopyridinyl, furopyridinyl (such as furo[2,3-c]pyridinyl, furo[3,2-b]pyridinyl, or furo[2,3-b]pyridinyl), imidazopyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, thienothienyl and the like. Preferably G¹ and G² may be (4-arylC₁₋₈alkyl)C₁₋₈alkyl. Particularly preferred compounds can be represented by Formula II:

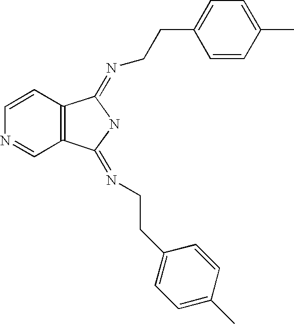

Unless specified otherwise, the terms “alkyl”, “alkenyl”, and “alkynyl,” whether used alone or as part of a substituent group, include straight and branched chains having 1 to 8 carbon atoms, or any number within this range. The term “alkyl” refers to straight or branched chain hydrocarbons. “Alkenyl” refers to a straight or branched chain hydrocarbon with at least one carbon-carbon double bond. “Alkynyl” refers to a straight or branched chain hydrocarbon with at least one carbon-carbon triple bound. For example, alkyl radicals include methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl.

“Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 3 of the hydrogen atoms thereon with aryl, heteroaryl, halogen, OH, CN, mercapto, nitro, amino, C₁-C₈-alkyl, C₂-C₈-alkenyl, C₁-C₈-alkoxyl, C₁-C₈-alkylthio, C₁-C₈-alkyl-amino, di(C_{1-C}₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Illustrative aryl radicals are described above.

Whether used alone or as part of a substituent group, “heteroaryl” refers to a cyclic, fully unsaturated radical having from five to ten ring atoms of which one ring atom is selected from S, O, and N; 0-2 ring atoms are additional heteroatoms independently selected from S, O, and N; and the remaining ring atoms are carbon. The radical may be joined to the rest of the molecule via any of the ring atoms. Exemplary heteroaryl groups are described above.

Co-crystal structure analysis of this preferred compound with PTP-1B reveals that one of the two symmetrical aryl side chains of the compound must bend out of the way in order for the interaction between the pyridine nitrogen and the binding pocket to occur. In so doing, the chain twists to form high energy gauche bonds, and experiences significant steric hindrance. Replacement of either or both of these preferred (4-arylC₁₋₈alkyl)C₁₋₈alkyl side chains would thus be expected to reduce steric hindrance, thereby increasing the strength of the interaction between the pyridine nitrogen of the compound(s) and the binding pocket of PTP-1B. Thus, replacement of the preferred aryl chains with smaller alkyl or aryl side chains would provide further exemplary preferred compounds. Examples of such alkyl or aryl side chains may include, but are not limited to, methyl, ethyl, iso-propyl, benzyl, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimetbylcarbamylphenyl, pyridinyl, pyrazinyl, pyrimidinyl, pyridazinyl, pyrrolyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, N-oxo-pyridyl, 1,1-dioxothienyl, benzothiazolyl, benzoxazolyl, benzothienyl, quinolinyl-N-oxide, benzimidazolyl, benzopyranyl, benzisothiazolyl, benzisoxazolyl, benzodiazinyl, benzofurazanyl, indazolyl, indolizinyl, benzofuryl, cinnolinyl, quinoxalinyl, pyrrolopyridinyl, furopyridinyl (such as furo[2,3-c]pyridinyl, furo[3,2-b]pyridinyl, or furo[2,3-b]pyridinyl), imidazopyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, thienothienyl and the like.

Metabolites of the inventive compounds that are prophylactically or therapeutically active are within the scope of the present invention, as are prodrugs, which are compounds that can be converted to the claimed active compounds or salts of the claimed active compounds after they have been administered to a subject. Furthermore, some of the compounds within the scope of the invention described herein contain one or more asymmetric centers and may thus give rise to diastereomers and enantiomers, which in turn can be resolved as optical isomers. The present invention includes all such diastereomers and enantiomers, including racemic mixtures and resolved enantiomerically pure forms, as well as the pharmaceutically acceptable salts thereof. Some of the compounds described herein may contain olefinic double bonds, and unless specified otherwise, include both E and Z geometric isomers. Any reference to the inventive compounds herein is meant to be inclusive of the metabolite and prodrug forms thereof, as well as any diastereomers, enantiomers, racemic mixtures, or resolved enantiomeric forms thereof, and their pharmaceutically acceptable salts.

Pharmaceutically acceptable salts of the inventive compounds are also contemplated to be within the scope of the present invention and as such, any reference to the inventive compounds is meant to be inclusive of the pharmaceutically acceptable salts thereof. Pharmaceutically acceptable salts can be prepared from pharmaceutically acceptable bases including inorganic bases and organic bases. Salts derived from inorganic bases include aluminum, ammonium, calcium, copper, ferric, ferrous, lithium, magnesium, manganic and manfanous salts, potassium, sodium, zinc, and the like. Non-limiting examples of salts derived from pharmaceutically acceptable organic bases include salts of primary, secondary and tertiary amines, substituted amines including naturally occurring substituted amines, cyclic amine and basic ion exchange resins, such as arginine, betaine, caffeine, choline, N,N′-dibenzylethylenediamine, diethylamine, 2-diethylaminoethanol, 2-dimethylaminoethanol, ethanolatnine, ethylenediamine, N-ethyl-morpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine and the like.

When the inventive compound is basic, salts may be prepared from pharmaceutically acceptable acids, including inorganic and organic acids. Such acids include acetic, adipic, aspartic, 1,5-napthalenedisulfonic, benzenesulfonic, benzoic, camphorsulfonic, citric, 1,2-ethanedisulfonic, ethanesulfonic, ethylenediaminetetraacetic, fumaric, glucoheptonic, gluconic, glutamic, hydroiodic, hydrobromic, hydrochloric, isothionic, lactic, maleic, malic, mandelic, methanesulfonic, mucic, 2-napthalenesulfonic, nitric, oxalic, pamoic, pantothenic, phosphoric, pivalic, propionic, salicylic, stearic, succinic, sulfuric, tartaric, p-toluenesulfonic, undecanoic, and the like.

The inventive compounds can be readily and easily synthesized by those of ordinary skill in the art using any of a wide variety of known synthetic schemes. The particular synthesis utilized will depend upon the compound synthesized and the particular method chosen is not critical. Rather, any inventive compound capable of the binding interaction described herein is considered to be within the scope of the present invention however synthesized. One illustrated method for synthesis of an illustrative inventive compound is provided hereinbelow, at Example 1.

Due at least in part to the hydrogen bond acceptor and the more positive aqueous phase free energy, compounds of the present invention bind to the primary binding pocket of PTP-1B, they are potent inhibitors of the same. In fact, the compound exemplified herein exhibits excellent efficacy, having an IC50 value of less than about 1 μM as determined by the assay described in Example 2. Preferred compounds according to the present invention are expected to be capable of providing inhibition activity of PTP-1B with inhibition % of at least about 80%, preferably at least about 85%, more preferably at least about 90% inhibition at 10 μM.

Those of ordinary skill in the art may readily and easily confirm the PTP-1B inhibition activity of the inventive compounds by any known method. For example, the inhibition effect of the inventive compound(s) can be measured by enzyme assays, e.g. as may be monitored via fluorescence, binding assays, e.g., as may include a Western blot using antiphosphotyrosine antibody, or, can be monitored via measuring the release of inorganic phosphate from the receptor using a malachite green assay, as described below in connection with Example 2, which would show decreased dephosphorylation of the phosphorylated insulin receptor by PTP-1B in the presence of the inventive compound.

The inhibition effect of the inventive compound(s) can also be measured in a cell-based assay. A phenotype associated with decreased PTP-1B activity, such as cell proliferation or cell motility, can be monitored in the presence or absence of the inventive compound(s). Also, gene fusion comprising a promoter that is sensitive to PTP-1B activity and a reporter gene that can be easily measured, such as Green Fluorescence Protein (GFP), can also be used for the cell-based assay. Further, the PTP-1B inhibition effect of the inventive compound(s) can also be tested and/or confirmed in vivo, using any of the number of strains of mice and rats commercially available for such use, via glucose and insulin tolerance tests well known to those of ordinary skill in the art. (see e.g., Elchebly et al., Science, 1999, 283: 1544).

The inventive compounds are advantageously employed to inhibit PTP-1B, and preferably are used in therapeutic or prophylactic methods of treating, preventing, or delaying the onset of conditions mediated by PTP-1B. PTP-1B is currently known to play a part in a wide variety of disease conditions and bodily functions, and is being investigated for its role in many others. As such, the particular disease, disorder or condition prevented or treated is not critical. Rather, any disease or condition wherein PTP-1B plays a role is likely to benefit, i.e., either be treated or prevented, via administration of the inventive compounds is considered to be within the scope of the inventive method. Non-limiting examples of disorders or conditions in which PTP-1B levels may desirably be mediated include, but are not necessarily limited to, disorders in glucose and lipid metabolism, such as type I diabetes, type II diabetes, impaired glucose tolerance, insulin resistance, obesity, hyperlipidemia, hypertriglyceridemia, hypercholesterolemia, dyslipidemia or low HDL levels; immune dysfunctions including inflammatory bowel disease and autoimmunity diseases; dysfunctions of the coagulation system; allergic diseases including asthma; osteoporosis, proliferative disorders including cancers such as prostate cancer, ovarian cancer, leukemias, adipose cell tumors, adipose cell carcinoma, liposarcoma, and breast cancer, diseases with increased or decreased syntheses of effects of growth factors, diseases with decreased or increased synthesis of growth factors or cytokines that regulate the release of/or response to growth factors, disease of the brain including Alzheimer's disease and schizophrenia; infectious diseases; nephropathy; neuropathy; retinopathy; artherosclerosis; vascular restenosis; pancreatitis; polycystic ovary syndrome, ischemia, hypertension, stroke, and heart disease. Due at least in part to the great need for additional, alternative therapies for the same, methods of treating disorders in glucose and lipid metabolism and proliferative disorders via administration of a therapeutically or prophylactically effective amount of the inventive compound are particularly preferred.

As mentioned above, the method of the present invention may be applied prophylactically or therapeutically, as desired or needed. That is, the present inventive method contemplates that the inventive compounds may be administered to a subject at risk of, or susceptible to, a disorder or condition mediated by PTP-1B, prior to the manifestation or presentation of any significant symptoms of the disease or condition in order to prevent or delay progression of the disease or condition. Or, treatment via the inventive method may be initiated after the onset of disease, i.e., the method may be utilized to therapeutically treat a subject suffering from symptoms associated with a disease or condition mediated by PTP-1B, so that said therapeutic agent desirably at least minimally inhibits PTP-1B activity. In either case, the methods provided by the present invention comprise administering either a prophylactically effective amount, or a therapeutically effective amount, as the case may be, to a subject in need of such treatment.

Methods are known in the art for determining therapeutically and prophylactically effective doses of the inventive compounds. Furthermore, and as is also understood by those of ordinary skill in the art, specific dose levels for any particular subject will depend upon a variety of factors including the age, body weight, general health, sex, diet, time of administration, route of administration, rate of excretion, any additional therapeutic agents administered in combination therewith and the severity of the disease or condition being treated. Generally speaking then, the prophylactic or therapeutic treatment of the above identified conditions is expected to be achieved via administration of dosage levels of the inventive compound in amounts from about 0.01 mg/kg to about 100 mg/kg, 0.03 mg/kg to about 75 mg/kg, 0.05 mg/kg to about 50 mg/kg body weight per day, or from about 0.1 mg/kg to about 10 mg/kg of body weight per day. Whatever the desired or appropriate dosage level, it may be administered in a single daily dose, or the total daily dosage may be administered in divided doses of two, three or four times daily. Alternatively, the dosage can be formulated to be delivered in a substantially continuous fashion, as may be provided by sustained and/or controlled release dosage forms, or by a transdermal patch.

The inventive compound(s), and any additional therapeutic agents if desirable and as described below, are typically desirably combined with one or more pharmaceutically acceptable carriers into a dosage unit or pharmaceutical composition for administration to a subject in need of such treatment and any such dosage units or pharmaceutical compositions are within the scope of the present invention. The amount of inventive compound to be included in such a dosage form will depend upon the patient being treated, the mode of administration and the desired delivered dose. Representative pharmaceutical compositions will generally include from about 0.01 mg to about 1000 mg, from about 1 mg to 800 mg, from about 50 mg to about 600 mg, or from about 100 mg to about 500 mg, of the inventive compound.

The present inventive method further contemplates that, in many instances, it may be desirable or necessary to administer a combination of prophylactic or therapeutic agents to a subject to treat the same, related, or different conditions. Any such combination therapies comprising any additional therapeutic agent and including one or more of the inventive compounds are considered to be within the scope of the invention. Where a second pharmaceutical is used in addition to the inventive compound the two pharmaceuticals may be administered together in a single composition, separately at approximately the same time, or separately on separate dosing schedules. All that is required is that the dosing schedules of the inventive compound and any desired additional therapeutic agent overlap in time and thus are being followed concurrently.

Examples of additional therapeutic agents that may desirably be included in a combination therapy with the inventive compound(s) include, but are not limited to:
(a) insulin sensitizers including (i) PPARy agonists such as the glitazones (e.g. troglitazone, pioglitazone, englitazone, MCC-555, rosiglitazone, and the like; (ii) biguanides such as metformin and phenformin;
(b) insulin or insulin mimetics;
(c) sulfonylureas such as tolbutamide and glipizide;
(d) α-glucosidase inhibitors (such as acarbose);
(e) cholesterol lowering agents such as (i) HMG-CoA reductase inhibitors (lovastatin, simvastatin and pravastatin, fluvastatin, atorvastatin, rivastatin), (ii) sequestrants (cholestyramine, colestipol and a dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and benzafibrate), (v) inhibitors of cholesterol absorption such as for example beta-sitosterol and acyl CoA:cholesterol acyltransferase inhibitors such as for example melinamide, and (vi) probucol;
(f) PPARα/γ agonists;
(g) anti-obesity compounds such as appetite suppressants, fenfluramine, dexfenfluramine, phentiramine, sulbitramine, orlistat, neuropeptide Y5 inhibitors (NP Y5 receptor antagonists), leptin, β3 adrenergic receptor agonists, and PPARγ antagonists and partial agonists;
(h) ileal bile acid transporter inhibitors;
(i) insulin receptor activators;
(j) anti-proliferative agents and agents useful for interfering with cell proliferation such as enoxaprin, andiopeptin, hirudin, acetylsalicylic acid, and thymidine kinase;
(k) angiogenic factors including growth factors such as acidic and basic fibroblast growth factors, vascular endothelial growth factor, epidermal growth factor, transforming growth factor α and β, platelet-derived endothelial growth factor, platelet derived growth factor, tumor necrosis factor α, hepatocyte growth factor and insulin like growth factor,
(l) anti-inflammatory agents such as desamethasone, prednisone, corticosterone, budesonide, estrogen, sulfasalazine, and mesalamine;
(m) antineoplastic/anti-miotic agents such as paclitaxel, 5-fluorouracil, cisplatin, vinblastine, vincristine, epothilones, endostatin, angiostatine, thymidine kinase inhibitors, herceptin, topoisomerase I inhibitors including camptothecin and its analogous, docetaxel, cisplatin, and gemcitabine;
(n) anesthetic agents such as lidocaine, bupivacaine, and ropivacaine; and
(O) anti-coagulants such as heparin, antithrombin compounds, platelet receptor antagonists, aspirin, prostaglandin inhibitors and platelet inhibitors.

In those embodiments of the invention wherein the condition being treated is diabetes I or II, particularly advantageous combination therapies may include additional therapeutic agents effective to further treat the diabetes, or other diseases, e.g., artherosclerosis, or conditions, e.g., obesity, that may be related to diabetes. Such advantageous combinations could thus include anti-diabetes compounds ((a)-(d), (f) and (i), above), anti-obesity compounds ((g), above) or compounds or compositions for lipid profile control, as may also be useful in the treatment of artherosclerosis ((e) and (h), above).

Whether administered alone or in combination with an additional therapeutic agent, the inventive compound may be administered by any known route of administration, including, orally, topically, parenterally (including subcutaneous, intravenous, intramuscular, and intrasternal injection or infusion administration techniques), by inhalation spray or rectally in dosage units or pharmaceutical compositions containing conventional pharmaceutically acceptable carriers and any such dosage units or pharmaceutical compositions are within the scope of the present invention.

Pharmaceutical compositions adapted for oral administration include solid forms such as pills, tablets, caplets, and hard or soft capsules (each including immediate release, timed release, and sustained release formulations) as well as lozenges and dispersible powders or granules. Liquid forms of pharmaceutical compositions adapted for oral administration include solutions, syrups, elixirs, emulsions, and aqueous or oily suspensions. Any of these dosage forms may be prepared according to any method or compounding technique known in the art for the manufacture of pharmaceutical compositions. Pharmaceutically acceptable carriers that may be desirably utilized in the manufacture of solid oral dosage forms include inert diluents, such as calcium carbonate, sodium carbonate, lactose, calcium phosphate or sodium phosphate; granulating or disintegrating agents, such as corn starch or alginic acid; binding agents, such as starch, gelatin, or acacia; and lubricating agents such as magnesium stearate, stearic acid or talc. If desired, solid pharmaceutical compositions adapted for oral administration may further include one or more sweetening agents, flavoring agents, coloring agents, or preserving agents in order to provide attractive or palatable preparations.

In those embodiments wherein the dosage form is a tablet or pill, it may either be uncoated or coated, and if coated, may be coated by any known technique. Further, the coating, if desirably provided, can be formulated or applied by known techniques so that the coating can delay disintegration of the tablet or pill, and thus, absorption of the inventive compound, thereby providing a controlled and/or sustained release dosage form capable of providing sustained therapeutic or prophylactic effect over a longer period. For example, the tablet or pill can comprise an inner dosage and an outer dosage component, the latter being in the form of an envelope over the former. An enteric layer that serves to resist disintegration in the stomach and permits the inner component to pass substantially intact into the duodenum or to be delayed in release can separate the two components. A variety of materials can be used for such enteric layers or coatings, including a number of polymeric acids, shellac, cetyl alcohol and cellulose acetate. Alternatively, in those embodiments wherein such a controlled and or sustained release is desired, tablets, pills or capsules may be formulated as osmotic pump dosage forms by any known method.

Pharmaceutical compositions adapted for oral administration may also be presented as hard or soft gelatin capsules, wherein the inventive compound may be mixed with an inert solid diluent, such as calcium carbonate, calcium phosphate or kaolin in the case of the former or with water or miscible solvents such as propylene glycol, PEG's and ethanol, or an oil medium such as peanut oil, liquid paraffin, or olive oil in the case of the latter.

Aqueous suspensions can be prepared that contain the inventive compound(s) in admixture with excipients suitable for the manufacture of aqueous suspensions. Such excipients include suspending agents, such as sodium carboxymethylcellulose, methylcellulose, hydroxypropylmethylcellulose, sodium alginate, polyvinyl-pyrrolidone, gum tragacanth and gum acacia, dextran, polyvinyl-pyrrolidone or gelatin; and dispersing or wetting agents such as lecithin, polyoxyethylene stearate, heptadecaethyleneoxycetanol, polyoxyethylene sorbitol monooleate, and polyethylene sorbitan monooleate. Aqueous suspensions may also contain one or more preservatives, such as ethyl or n-propyl, p-hydroxybenzoate; one or more coloring agents, one or more flavoring agents, and one or more sweetening agents, such as sucrose, saccharine or aspartame.

Oily suspensions may be formulated by suspending the inventive compound(s) in a vegetable oil, such as cottonseed, olive, sesame or coconut oil, or in a mineral oil, such as liquid paraffin. The oily suspensions may contain a thickening agent, such as beeswax, hard paraffin, or cetyl alcohol. Sweetening agents such as those set forth above, and flavoring agents may be added to provide a palatable oral preparation. Such oily suspensions may be preserved by the inclusion of an anti-oxidant such as ascorbic acid.

Dispersible powders and granules suitable for the preparation of an aqueous suspension suitable for oral administration can provide the inventive compound(s) in admixture with a dispersing or wetting agent, suspending agent, and one or more preservatives, all of which have been discussed above. Sweetening, flavoring, or coloring agents may also be present, if desired.

Pharmaceutical compositions suitable for oral administration may also be presented in the form of an oil-in-water emulsion. The oily phase may be a vegetable or mineral oil, such as those described above, or mixtures of these. Suitable emulsifying agents may be naturally-occurring phosphatides, such as soy bean, lecithin, sorbitan monooleate, or polyoxyethylene sorbitan monooleate. The emulsions may also contain sweetening or flavoring agents.

Syrups and elixirs may be formulated with sweetening agents, for example, glycerol, propylene glycol, sorbitol or sucrose. Such formulations may also contain a demulcent, a preservative and flavoring or coloring agents.

The pharmaceutical compositions may be further provided in a form adapted for parenteral administration, i.e., by injection or infusion. Injectable aqueous or oleaginous suspensions are desirably sterile and may be formulated according to known methods using suitable dispersing, wetting and suspending agents as mentioned above. A parenterally-acceptable diluent or solvent may also be utilized, such as 1,3-butanediol, water, Ringer's solution, and isotonic sodium chloride. Cosolvents such as ethanol, propylene glycol or polyethylene glycols may also be used. In addition, sterile, fixed oils are conventionally employed as solvents or suspending mediums in injectable or infusible solutions, and these may include any bland fixed oil, such as any of the synthetic mono- or diglycerides. Fatty acids such as oleic acid also may be utilized in the preparation of injectable or infusible solutions.

The pharmaceutical composition may also be presented in the form of a suppository. Suppositories can be formulated by mixing the inventive compound(s) and any additional desired therapeutic agent(s) with a suitable non-irritating excipient that is solid at room temperature but molten at body temperature, thereby releasing the inventive compound(s). Suitable materials include cocoa butter and polyethylene glycols.

For topical use, creams, ointments, gels, solutions or suspensions containing the inventive compound(s) may be prepared. As used herein, topical use includes mouth washes and gargles. Topical formulations may include cosolvents, emulsifiers, penetration enhancers, preservatives, emollients, and the like.

The inventive compound of Formula I can also be provided in a pharmaceutical composition in the form of liposome delivery systems, such as small unilamellar vesicles, large unilamellar vesicles, and multilamellar vesicles. Liposomes can be formed from a variety of lipids, including but not limited to amphipathic lipids such as phosphatidylcholines, sphingomyelins, phosphatidylethanolamines, phophatidylcholines, cardiolipins, phosphatidylserines, phosphatidylglycerols, phosphatidic acids, phosphatidylinositols, diacyl trimethylammonium propanes, diacyl dimethylammonium propanes, and stearylamine, neutral lipids such as triglycerides, and combinations thereof. They may either contain cholesterol or may be cholesterol-free.

This invention will be better understood by reference to the schemes and examples that follow, but those skilled in the art will readily appreciate that these are only illustrative of the invention as described more fully in the claims which follow thereafter. Either elemental analysis or NMR confirms the structures of the compounds, where peaks assigned to characteristic protons in the inventive compounds are presented where appropriate. ¹H NMR shifts (δ_{H}) are given in parts per million (ppm) down field from tetramethylsilane as internal reference standard.

### EXAMPLE 1

### Synthesis of 1,3-bis-(2-p-tolyl-ethylamino)-2,3-dihydro-1H-pyrrolo [3,4-C]pyridine

A mixture of 3,4-pyridinedicarbonitrile (0.5g, 3.8 mmol), 4-methylphenethylamine (1.54g, 11.4 mmol, 3 equivalents) and elemental sulfur (100 mg) was warmed to 40° C. under nitrogen. After 10 h, the resulting mixture was allowed to cool to 25° C., diluted with ethyl acetate (100 mL), and the product was collected by filtration. Recrystallization from aqueous ethanol afforded pure 1,3-Bis-(2-p-tolyl-ethylimino)-2,3-dihydro-1H-pyrrolo[3,4-c]pyridine (900 mg, 62%) as a yellow solid. ¹H (400 MHz, CDCl₃): 9.13 (s, 1H), 8.78 (d, 1H, J=7.5 Hz), 7.82 (d, 1H, J=7.9 Hz), 7.25 (d, 4H, J=7.5 Hz), 7.05 (d, 4H, J=7.5 Hz), 4.01-3.85 (m, 4H), 2.95 (m, 4H), 2.25 (s, 6H).

### EXAMPLE 2

### Assay for PTP-1B Phosphatase Activity

PTP-1B phosphatase activity was measured as its ability to dephosphorylate a phosphorylated insulin receptor. In particular, a phosphorylated peptide derived from insulin receptor was used for the assay. The dephosphorylation reaction was monitored using a malachite green assay (Lanzetta et al., Anal Biochem. 1979, 100: 95-7), which measures the release of inorganic phosphate from the activated insulin receptor or the phosphorylated peptide derived from insulin receptor. A PTP-1B inhibitor decreases the yield of inorganic phosphate when it is added to the assay.

Materials

A substantially purified, soluble, truncated human PTP-1B GST fusion protein was used as the source of PTP-1B enzyme. The fusion protein, which lacks the carboxyl terminus 35 amino acid residues of PTP-1B, was recombinantly expressed and purified from Escherichia coli host cells according to instructions for the pGEX kit from Pharmacia Biotech Inc. The phosphorylated kinase domain of insulin receptor-1 (Anaspec, Cat No: 20272, with the sequence of H-Thr-Arg-Asp-Ile-pTyr-Glu-Thr-Asp-pTyr-pTyr-Arg-tys-OH) was used as the substrate. The assay buffer consisted of: 33 mM Tris pH7.4, 2 mM EDTA, and 0.1% BSA. The malachite green stock solution was prepared by dissolving malachite green in 4N HCl to a final concentration of 1.52 mg/ml. The ammonium malybdate stock solution was prepared by dissolving ammonium malybdate in H₂O to a final concentration of 40 mg/ml followed by filtering the solution. The malachite green and ammonium malybdate solution was prepared by mixing equal volumes of the malachite green and the ammonium malybdate stock solutions, and adding tween-20 (0.074%, final concentration) to the mixture.

Procedure

PTP-1B GST fusion protein (10 ng in 48 μl of assay buffer) was mixed with the phosphorylated kinase domain of insulin receptor-1 (70 μM in 201 μl of assay buffer) and the test compound (at desired concentration in 2 μl DMSO). The reaction mixture was incubated at room temperature for about 15 minutes. Then, 30 μl of malachite green and ammonium malybdate solution was added to the reaction mixture. After additional 30 minutes incubation at room temperature, the amount of inorganic phosphate released from the phosphorylated kinase domain of insulin receptor-1 in the reaction mixture was measured by the absorbance at 650 nm.

A PTP-1B inhibitor will decrease the release of inorganic phosphate when the inhibitor is added to the reaction mixture. The inhibition % of a test compound on PTP-1B is defined as the amount of inorganic phosphate in the reaction comprising the test compound divided by the amount of inorganic phosphate in the reaction without the test compound. IC₅₀ of an individual inhibitor refers to the concentration of the inhibitor at which the amount of inorganic phosphate is reduced by one-half as compared with reactions containing no inhibitor. It was found that the compound of Formula II inhibited PTP-1B phosphatase activity with an IC₅₀ of 0.9096 μM under the assay conditions described herein.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A pharmaceutical composition of claim 1, wherein the compound has a formula according to Formula (I): 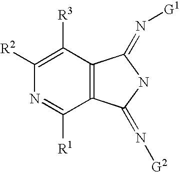
or an optical isomer, enantiomer, diastereomer, racemate or racemic mixture thereof, ester, prodrug or metabolite form, or a pharmaceutically acceptable salt thereof, wherein R¹, R² and/or R³ are independently hydrogen atoms or alkyl groups and G¹ and G² are aryl, heteroaryl, alkyl, arylalkanyl, arylalkenyl, arylalkynyl, heteroarylalkanyl, heteroarylalkenyl, or heteroarylalkynyl moieties.

2. The pharmaceutical composition of claim 1, wherein the compound has a formula according to Formula I and G₁ and G₂ are (4-arylC₁₋₈alkyl)C₁₋₈alkyl.

3. The pharmaceutical composition of claim 1, wherein the compound is: 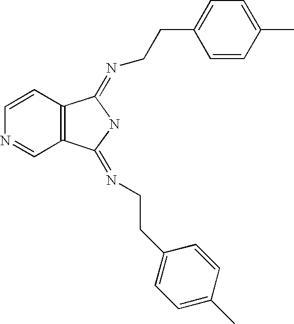
or an optical isomer, enantiomer, diastereomer, racemate or racemic mixture thereof, ester, prodrug or metabolite form, or a pharmaceutically acceptable salt thereof.

4. The pharmaceutical composition of claim 1, wherein the compound of formula I is provided in a therapeutically effective amount.

5. The pharmaceutical composition of claim 1, wherein the compound of formula I is provided in a prophylactically effective amount.

6. The pharmaceutical composition of claim 1, wherein the composition is adapted for administration as a part of a combination therapy.

7. The pharmaceutical composition of claim 1, wherein the composition is adapted for combination therapy via the inclusion therein of an additional therapeutic agent.

8. The pharmaceutical composition of claim 1, wherein the composition is adapted for combination therapy via the provision in connection therewith a description of a dosing regimen including administration of an additional pharmaceutical composition comprising an additional therapeutic agent.

9. A method of treating a subject suffering from a condition mediated by PTP-1B, comprising administering to said subject a therapeutically effective amount of a pharmaceutical composition comprising a compound capable of interacting with the primary binding pocket of PTP-1B and having a formula according to Formula I: 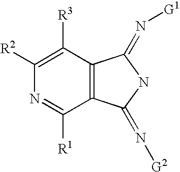
or an optical isomer, enantiomer, diastereomer, racemate or racemic mixture thereof, ester, prodrug or metabolite form, or a pharmaceutically acceptable salt thereof, wherein R¹, R² and/or R³ are independently hydrogen atoms or alkyl groups and G¹ and G² are aryl, heteroaryl, alkyl, arylalkanyl, arylalkenyl, arylalkynyl, heteroarylalkanyl, heteroarylalkenyl, or heteroarylalkynyl moieties.

10. The method of claim 9, wherein G₁ and G₂ are (4-arylC₁₋₈alkyl)C₁₋₈alkyl.

11. The method of claim 10, wherein the compound is: 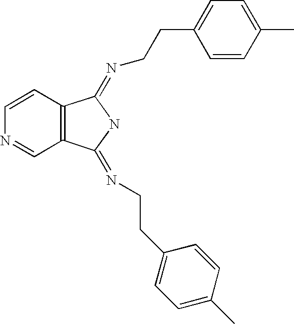
or an optical isomer, enantiomer, diastereomer, racemate or racemic mixture thereof, ester, prodrug or metabolite form, or a pharmaceutically acceptable salt thereof.

12. The method of claim 9, wherein the step of administering comprises administering a pharmaceutical composition comprising the compound of Formula I.

13. The method of claim 9, wherein the pharmaceutical composition comprises an additional therapeutic agent.

14. The method of claim 13, wherein the additional therapeutic agent comprises an anti-diabetes compound, anti-obesity compounds, a compound for lipid profile control, or a combination of these.

15. The method of claim 13, wherein the additional therapeutic agent comprises an anti-cancer agent.

16. A method of inhibiting in a subject the onset of a disorder related to PTP-1B, comprising administering to said subject a prophylactically effective amount of a pharmaceutical composition comprising a compound capable of interacting with the primary binding pocket of PTP-1B and wherein the compound has a formula according to Formula I: 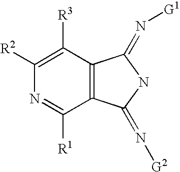
or an optical isomer, enantiomer, diastereomer, racemate or racemic mixture thereof, ester, prodrug or metabolite form, or a pharmaceutically acceptable salt thereof, wherein R¹, R² and/or R³ are independently hydrogen atoms or alkyl groups and G¹ and G² are aryl, heteroaryl, alkyl, arylalkanyl, arylalkenyl, arylalkynyl, heteroarylalkanyl, heteroarylalkenyl, or heteroarylalkynyl moieties.

17. The method of claim 16, wherein G₁ and G₂ are (4-arylC₁₋₈alkyl)C₁₋₈alkyl.

18. The method of claim 17, wherein the compound is: 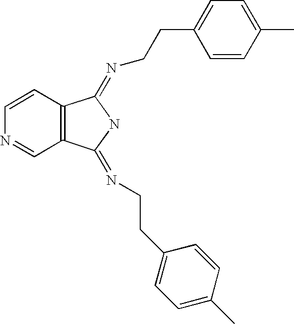
or an optical isomer, enantiomer, diastereomer, racemate or racemic mixture thereof, ester, prodrug or metabolite form, or a pharmaceutically acceptable salt thereof.

19. The method of claim 16, wherein the step of administering comprises administering a pharmaceutical composition comprising the compound of Formula I.

20. The method of claim 19, wherein the pharmaceutical composition comprises an additional therapeutic agent.

21. The method of claim 20, wherein the additional therapeutic agent comprises an anti-diabetes compound, anti-obesity compounds, a compound for lipid profile control, or a combination of these.

22. The method of claim 20, wherein the additional therapeutic agent comprises an anti-cancer agent.

23. The method of claim 9, wherein said disorder is a disorder in glucose and lipid metabolism.

24. The method of claim 9, wherein said disorder in glucose and lipid metabolism is hypertension, type I Diabetes Mellitus, or a condition of reduced insulin sensitivity.

25. The method of claim 9, wherein said condition of reduced insulin sensitivity is type 2 Diabetes Mellitus or obesity.

26. A method of inhibiting PTP-1B comprising contacting a compound capable of interacting with the primary binding pocket of PTP-1B via a hydrogen bond acceptor and having an aqueous phase free energy more positive than about −1200 kJ/mol with a medium comprising PTP-1B, thereby causing the compound to bind with PTP-1B thereby inhibiting the activity of PTP-1B.

27. The method of claim 26, wherein the medium comprising PTP-1B is a cell.

28. The method of claim 26, wherein the compound has an aqueous phase free energy more positive than about −400 kJ/mol.

29. The method of claim 26, wherein the compound has an aqueous phase free energy more positive than about −60 kJ/mol.

30. The method of claim 26, wherein the compound has a formula according to Formula: 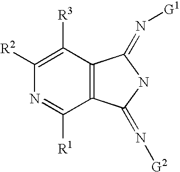
or an optical isomer, enantiomer, diastereomer, racemate or racemic mixture thereof, ester, prodrug or metabolite form, or a pharmaceutically acceptable salt thereof, wherein R¹, R² and/or R³ are independently hydrogen atoms or alkyl groups and G¹ and G² are aryl heteroaryl, alkyl arylalkanyl, arylalkenyl, arylalkynyl, heteroarylalkanyl, heteroarylalkenyl, or heteroarylalkynyl moieties.

31. The method of claim 30, wherein the compound is: 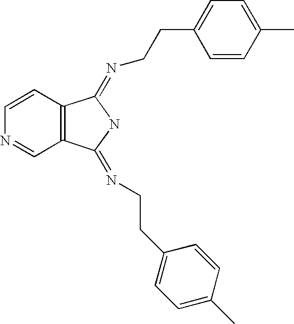
or an optical isomer, enantiomer, diastereomer, racemate or racemic mixture thereof, ester, prodrug or metabolite form, or a pharmaceutically acceptable salt thereof.
